(19) United States
(12) Plant Patent Application Publication
(10) Pub. No.: US 2026/0137014 P1
(43) Pub. Date: May 14, 2026

(54) MANDARIN PLANT NAMED 'RBA 13-18'

(50) ;

(71) Applicant: Florida Foundation Seed Producers, Inc., Marianna, FL (US)

(72) Inventor: Frederick GMITTER, JR., Lakeland, FL (US)

(21) Appl. No.: 19/381,534

(22) Filed: Nov. 6, 2025

Related U.S. Application Data
(60) Provisional application No. 63/719,056, filed on Nov. 11, 2024.

(51) Int. Cl. A01H 6/78; A01H 5/08

(52) U.S. Cl. CPC A01H 6/785; A01H 5/08
USPC PLT/202

(57) ABSTRACT

A new and distinct cultivar of orange plant named ‘RBA 13-18’, a complex mandarin hybrid, with high tolerance to CLas, vigor in the presence of Huanglongbing (HLB), and producing fruit and juice deemed useful to the processed sweet orange juice industry.

### CROSS-REFERENCE TO RELATED APPLICATIONS

This application claims the benefit of U.S. Provisional Patent Application No. 63/719,056, filed Nov. 11, 2024, entitled MANDARIN PLANT NAMED ‘RBA 13-18’, which is incorporated by reference herein in its entirety.

### ACKNOWLEDGEMENT OF FEDERAL RESEARCH SUPPORT

N/A.

Genus and species: Citrus reticulata hybrid.

Cultivar denomination: ‘RBA 13-18’.

### BACKGROUND OF THE NEW CULTIVAR

The present invention relates to a new and distinct cultivar of Citrus (mandarin), botanically known as Citrus reticulata hybrid and hereinafter referred to by the name ‘RBA 13-18’. This Citrus reticulata hybrid is a member of Rutaceae.

This cultivar arose from a cross of ‘LB8-9’ (U.S. Plant Pat. No. 21,356) x ‘Ortanique’ (a publicly available cultivar of unknown parentage, presumed to be a hybrid of some mandarin selection with sweet orange). The cross was made in 1993 near Lake Alfred, Florida, and a seedling tree was planted on property that was sold in 2000, at which time the original seedling tree was moved to another Citrus growing block near Haines City, Florida. The tree was maintained at this location and in the mid-2010s was identified as a survivor of multiple years of Huanglongbing (HLB) pressure. In 2020, CLas-infected budwood from the field grown tree was collected under permit from the Florida Department of Agriculture and Consumer Services, Division of Plant Industry (FDACS-DPI), and topworked onto five-year-old ‘Valencia’ (not patented) sweet orange trees on ‘Swingle’ (not patented) rootstock growing in an industry-led interinstitutional field trial on private property in Hardee County, Florida. Trees of ‘RBA 13-18’ were found to be in good health and fruit were collected and characterized, and they were found to be suitable for processing and blending into orange juice.

### SUMMARY OF THE INVENTION

The original tree of the new cultivar ‘RBA 13-18’ has shown notable HLB tolerance, as have various top-worked trees in a different location. The fruit of ‘RBA 13-18’ have been shown to be equivalent to sweet orange in size with acceptable firmness to allow typical industrial harvest and transport practice to be employed. The juice has high Brix values compared with common sweet oranges grown in the current HLB environment in Florida. Juice color scores exceed USDA grade A standards, and limonin content is low, thus minimizing or avoiding any bitterness or other off flavors developing in processed juice. Finally, internal taste panels have scored the processed and stored juice quality high and remarked that the flavor is similar to sweet orange juice. Thus, this selection has good potential to be a supplemental source to improve the orange juice end product being produced currently in Florida using fruit grown only in Florida.

There are currently no data supporting long-term field performance or yield for the new cultivar ‘RBA 13-18’. However, there is a high degree of confidence in the HLB tolerance of this cultivar and its ability to produce fruit with qualities that will enable their use in the orange juice processing stream, at the 10% maximum currently allowed under law.

The new cultivar ‘RBA 13-18’ can be compared to the commercial cultivars ‘LB8-9’ and ‘Ortanique’, the parents. Fruit of the new cultivar ‘RBA 13-18’ are distinctly different from those of both ‘LB8-9’ and ‘Ortanique’. Fruit of ‘LB8-9’ are globose, but usually with a conspicuous neck, and they are deep orange at maturity. Fruit of the new cultivar ‘RBA 13-18’, while globose, lack the neck that is typical of ‘LB8-9’, as well as being less deeply colored externally. Fruit of ‘Ortanique’ are broadly pyriform in shape, have conspicuous oil glands, and mature in March, whereas those of ‘RBA 13-18’ are globose, lack conspicuous oil glands, and mature from December to mid-February in central Florida.

### DESCRIPTION OF THE FIGURES

This new mandarin hybrid ‘RBA 13-18’ is illustrated by the accompanying photographs which show the tree's from, foliage and fruit. The colors shown are as true as can be reasonably obtained by conventional photographic procedures.

FIG. 1 shows whole fruit of the new cultivar ‘RBA 13-18’;

FIG. 2 shows cut and whole fruit of ‘RBA 13-18’;

FIG. 3 cut fruit of ‘RBA 13-18’, with a ruler shown for size comparison;

FIG. 4 shows cut fruit of ‘RBA 13-18’;

FIG. 5 shows a top-worked tree of ‘RBA 13-18’;

FIG. 6 shows a close-up view of an off-bloom flower and leaves on a tree of ‘RBA 13-18’ (photograph taken in June 2024);

FIG. 7 shows a shoot apex of a branch of ‘RBA 13-18’;

FIG. 8 shows branching habit of ‘RBA 13-18’;

FIGS. 9 and 10 show petals, anthers, and filaments of a flower of ‘RBA 13-18’, with a ruler shown for size estimates;

FIG. 11 shows pedicle and pistil structures of a flower of ‘RBA 13-18’, with a ruler shown for size estimates;

FIG. 12 shows an unopened flower bud of ‘RBA 13-18’ from an off bloom in June 2024, with a ruler shown for size estimates;

FIG. 13 shows an abaxial surface of a leaf of ‘RBA 13-18, with a ruler shown for size estimates; and

FIG. 14 shows an adaxial surface of a leaf of ‘RBA 13-18, with a ruler shown for size estimates.

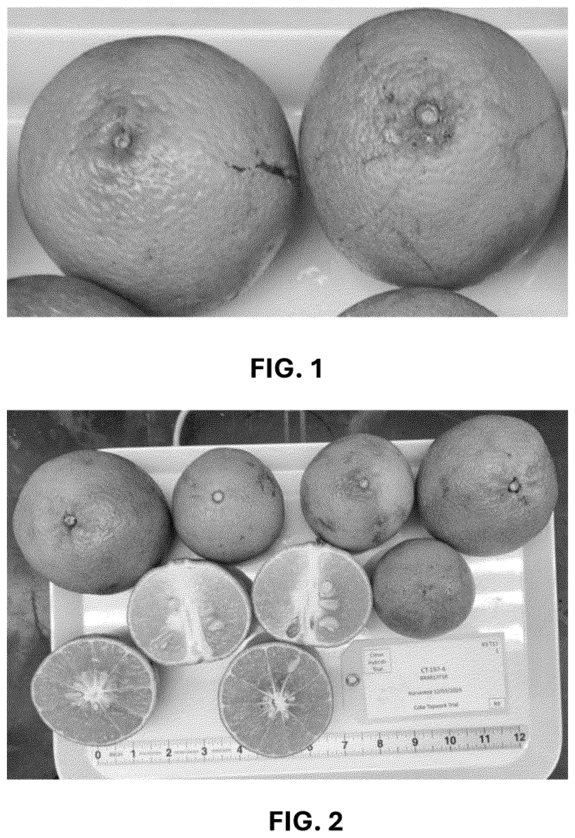

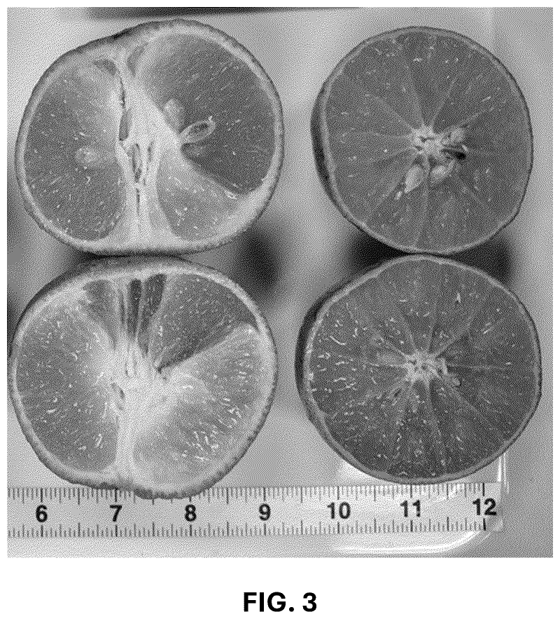

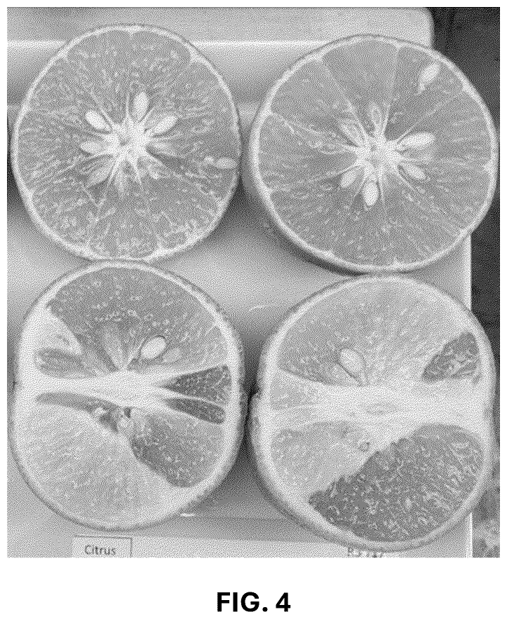

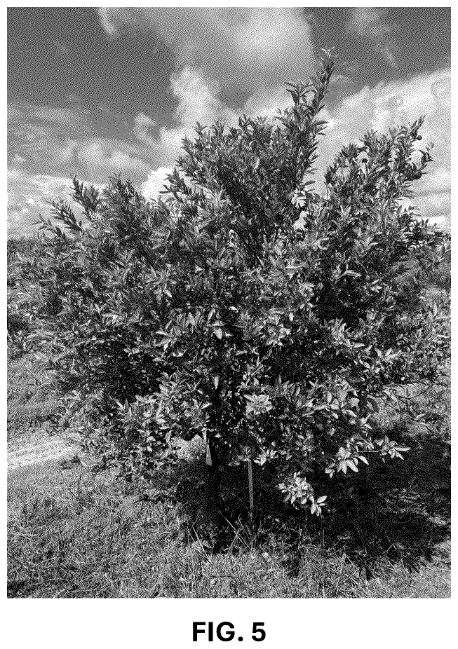

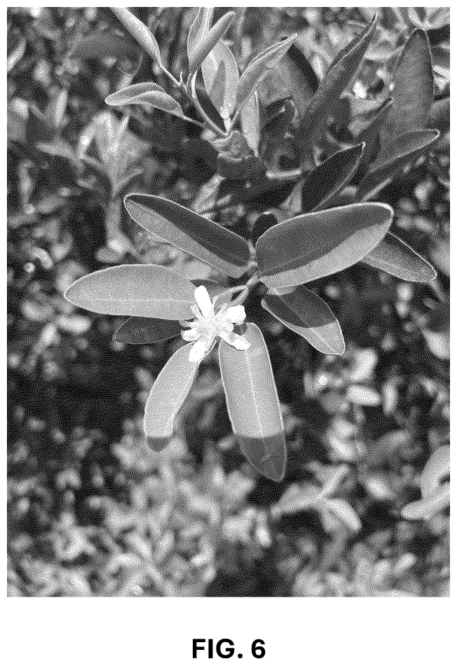

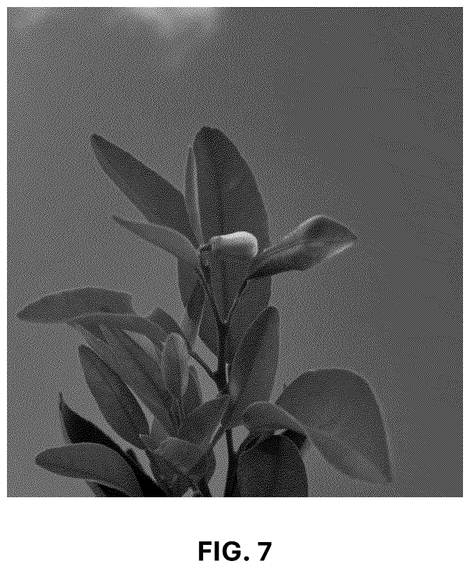

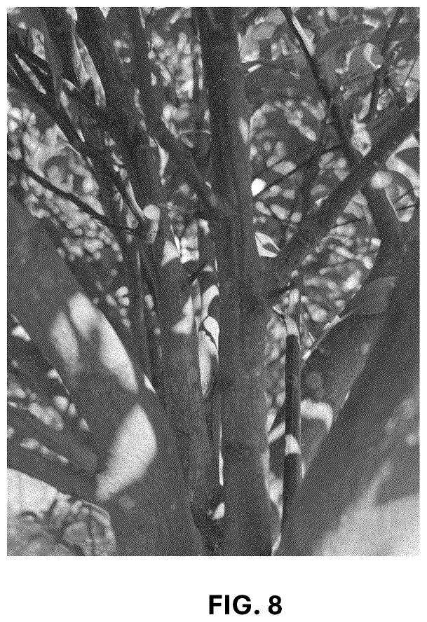

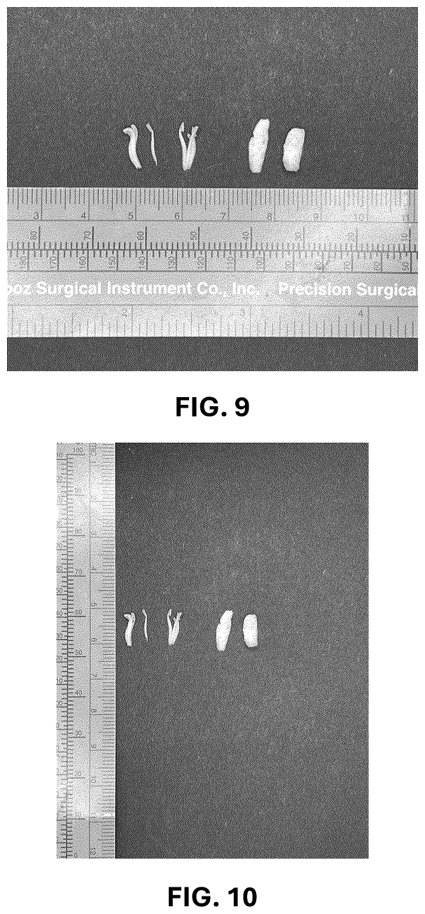

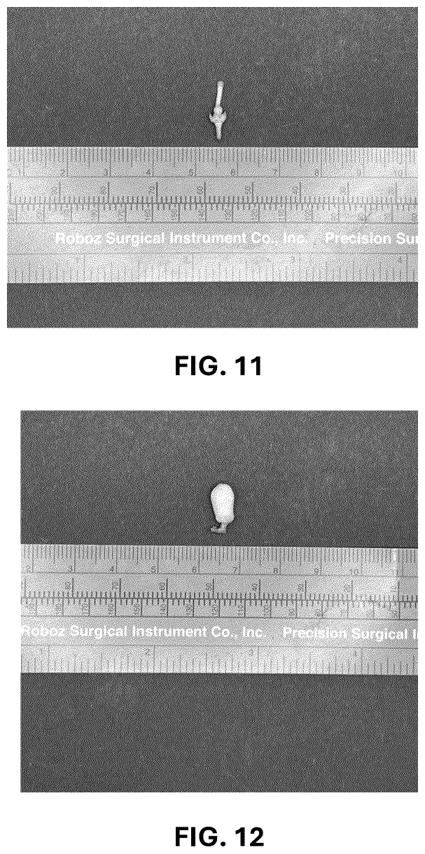

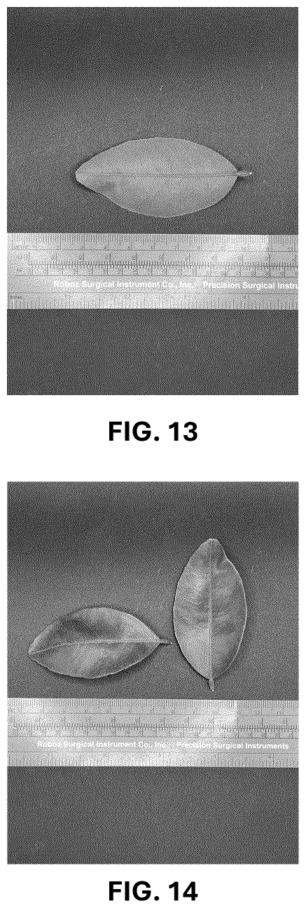

### DETAILED BOTANICAL DESCRIPTION OF THE CULTIVAR

The description below is based on data collected from one of these top-worked trees, from which budwood were harvested and entered into the FDACS-DPI Citrus Budwood Registration Bureau for shoot tip grafting and certification. The colors (except those in common terms) are described from R.H.S. Colour Chart published by The Royal Horticultural Society in London (2007, 5^{th} edition), in association with the Flower Council of Holland.

### Botanical Description

Botanical classification:
   Family.—Rutaceae.
   Botanical name.—Citrus reticulata hybrid.
   Common name.—Complex mandarin introgression hybrid.
   Cultivar.—‘RBA 13-18’
Parentage: ‘LB8-9’ x ‘Ortanique’.
Tree:
   Ploidy.—Diploid.
   Size.—Medium.
   Width across row.—2.13 meters on average.
   Width down Row.—2.4 meters on average.
   Tree height.—2.59 meters on average.
   Tree canopy diameter.—6.4 meters on average.
   Vigor.—High.
   Density.—High.
   Tree shape (form).—Round.
   Growth habit (current season).—Rounded.
Trunk:
   Trunk diameter.—32 cm at 30 cm above the ground on average.
   Trunk texture.—Rough.
   Trunk bark color.—RHS 199A (Grey-Brown Group).
Branches:
   Branch length.—1 m on average.
   Branch diameter.—30 cm on average.
   Crotch angle.—On average less than 90 degrees in the middle of the tree and some on the outside are more than 90 degrees (110-115 degrees).
   Branch texture.—Less rough than the trunk.
   Branch color.—RHS 199D (Grey-Brown Group).
Leaves:
Size (lamina average):
   Length.—77.2 mm on average.
   Width.—33.5 mm on average.
   L/W ratio.—2.30 on average.
   Thickness.—Relatively thick.
   Type.—Simple (unifoliate).
   Shape.—Elliptical.
   Apex.—Acute.
   Base.—Acute.
   Margin.—Serrulate. Leaf edges with smaller, more evenly spaced serrations than a serrated leaf.
Surface:
   Upper surface.—Smooth.
   Lower surface.—Smooth.
Color:
   Upper surface (adaxial).—RHS 137A Green Group.
   Lower surface (abaxial).—RHS 138A Green Group.
Petiole:
   Shape and attachment.—Brevipetiolate. The junction between petiole and lamina is articulate.
   Shape (petiole wing).—Small and narrow.
   Length.—10 mm on average.
   Width.—1.5 mm on average.
   Color.—RHS 137A (Green Group).
Flowers (based on single off-bloom found in June 2024):
   Type.—Hermaphrodite.
   Flowers bearing.—Single or clusters.
   Flower diameter.—20 mm on average.
   Flower depth.—12 mm on average.
   Flower blooming period.—Mid-late February through late March, depending on location and tree age.
   First bloom.—Mid-February.
   Full bloom.—Early March.
   Fragrance.—Fragrant.
   Length.—1 mm on average for the initial visible flower bud; 10 mm on average for the mature flower buds before opened.
   Diameter.—1 mm on average for the initial visible flower bud; 6 mm on average in the matured bud.
   Flower bud shape.—Round.
   Flower bud color.—RHS 142D (Green Group) for the initial visible flower bud; RHS 155A (White Group) for mature buds.
   Fertility.—Appears self-fertile.
Petals:
   Number.—5-6.
   Petal length.—9 mm on average.
   Petal width.—3 mm on average.
   Petal shape.—Recurved/cruciform.
   Apex shape.—Smooth with an obtuse angle.
   Base shape.—Obtuse.
Petal color:
   Upper surface.—RHS 155D (White Group).
   Lower surface.—RHS 155B (White Group).
   Margin.—Smooth.
Sepal:
   Number (per flower).—5.
   Shape.—Delta-shaped with an acute angle at the apex.
   Length.—1.6 mm.
   Width.—1.8 mm.
   Apex shape.—Acute.
   Margin.—Smooth.
Color:
   Upper surface.—RHS 145B (Yellow-Green Group).
   Lower surface.—RHS 145A (Yellow-Green Group).
Pedicel:
   Length.—12 mm on average.
   Diameter.—1.5 mm on average.
   Color.—RHS 144C (Yellow-Green Group).
Stamen:
   Number.—23 per flower on average.
   Length.—9 mm on average.
Anther:
   Length.—0.6 mm on average.
   Width.—0.2 mm on average.
   Color.—RHS 162B (Greyed-Yellow).
   Pollen color (general).—RHS 16B (Yellow-Orange Group).
Pistil:
   Number.—1.
   Length.—13 mm on average.
   Color.—RHS 150D (Yellow-Green Group).
   Style length.—6 mm on average.
   Style diameter.—1 mm on average.
   Style color.—RHS 150D (Yellow-Green Group).
   Ovary shape.—Oval.
   Ovary diameter.—2 mm on average.
   Ovary color.—RHS 154 (Yellow-Green Group).
Fruit:
   Size.—Similar or slightly larger than typical sweet orange.
   Length (height).—6-8 cm on average.
   Diameter.—7-8 cm on average.
   Shape.—Globose.
   Shape (cross-section).—Round.
   Apex.—Somewhat flattened apex.
   Base.—Rounded.
Harvesting:
   Date of first pick.—Mid-December.
   Date of last pick.—End of January to early February.
Rind:
   Adherence.—Adherent.
   Thickness.—1.5-2.5 mm on average.
   Texture.—Firm.
   Surface texture.—Slightly pebbled.
Color:
   Flavedo (epicarp).—RHS 17A (Yellow-Orange Group).
   Albedo (mesocarp).—RHS 158D (Yellow-White Group).
   Style end.—Closed.
   Rind oil cell density.—68 cells/cm².
   Oil gland size.—0.5 mm on average.
Flesh:
   Number of segments.—9-13 on average.
   Segment length.—6.5 cm on average.
   Segment width.—3.3 cm on average.
   Color.—Can vary depending on the harvest season from RHS 17A (Yellow-Orange Group) to RHS 23A (Yellow-Orange Group).
   Texture.—Soft.
Vesicles:
   Thickness.—Medium.
   Length.—8-12 mm on average.
   Diameter.—2 mm on average.
Juice quality:
   Limonin.—1.6.
   Average flavor score.—35 (high) and rated “similar to sweet orange.”
   Brix.—11.7 to 13.9.
   Acidity (average).—1.0.
   Ratio.—11-13.
   Juice color.—37.5.
Seeds:
   Presence.—Present.
   Fruit parthenocarpy.—No.
Resistance to disease: Highly ranked for HLB tolerance in the original tree, as well as repropagated trees in the top work trial; 95th percentile Normalized Difference Vegetation Index (NDVI, a remote sensing tool to monitor vegetation health) among more than 200 accessions.

1. A new and distinct mandarin plant named ‘RBA 13-18’ as illustrated and described herein.
